(19) United States
(12) Patent Application Publication
(10) Pub. No.: US 2010/0234688 A1
(43) Pub. Date: Sep. 16, 2010

(54) ACCESS PORT INCLUDING MULTI-LAYER SEAL AND SUTURE PARKS

(75) Inventor: Sally Carter, Wallingford, CT (US)

Correspondence Address: Tyco Healthcare Group LP, 60 MIDDLETOWN AVENUE, NORTH HAVEN, CT, 06473, US

(73) Assignee: Tyco Healthcare Group LP

(21) Appl. No.: 12/697,566

(22) Filed: Feb. 1, 2010

Related U.S. Application Data
(60) Provisional application No. 61/158,824, filed on Mar. 10, 2009.

(51) Int. Cl. A61B 1/32

(52) U.S. Cl. 600/208

(57) ABSTRACT

A seal assembly configured for use with a cannula assembly includes a housing and a suture retaining member disposed in mechanical cooperation with the housing. The suture retaining member is configured to hold at least one suture and configured to help prevent the tangling of sutures. A slit seal member is disposed in mechanical cooperation with the housing. The slit seal member is configured to create a substantially fluid-tight seal in the absence of a surgical instrument inserted therethrough. An instrument seal member is disposed in mechanical cooperation with the housing. The instrument seal member is configured to create a substantially fluid-tight seal around a surgical instrument inserted therethrough.

### CROSS REFERENCE TO RELATED APPLICATION

The present application claims the benefit of and priority to U.S. Provisional Application Ser. No. 61/158,824 filed on Mar. 10, 2009, the entire contents of which are incorporated herein by reference.

### BACKGROUND

1. Technical Field

The present disclosure relates to access ports for providing access into a patient's body. In particular, the present disclosure relates to access ports which are adapted to allow the introduction of surgical instrumentation into a patient's body. The access ports also include suture retaining members configured to hold sutures.

2. Description of the Related Art

In laparoscopic procedures surgery is performed in the interior of the abdomen through a small incision; in endoscopic procedures surgery is performed in any hollow viscus of the body through a narrow tube or cannula inserted through a small entrance incision in the skin. Laparoscopic and endoscopic procedures generally require that any instrumentation inserted into the body be sealed, i.e. provisions must be made to ensure that gases do not enter or exit the body through the incision as, for example, in surgical procedures in which the surgical region is insufflated. Moreover, laparoscopic and endoscopic procedures often require the surgeon to act on organs, tissue, and vessels far removed from the incision, thereby requiring that any instruments used in such procedures be relatively long and narrow.

For such procedures, the introduction of a tube into certain anatomical cavities such as the abdominal cavity is usually accomplished by use of a trocar assembly made up of a cannula assembly and an obturator assembly. Since the cannula assembly provides a direct passage for surgical instrumentation from outside the patient's body to access internal organs and tissue, it is important that the cannula assembly maintain a relatively gas-tight interface between the abdominal cavity and the outside atmosphere. The cannula assembly generally includes a cannula attached to a cannula housing containing a seal assembly adapted to maintain a seal across the opening of the cannula housing.

Since surgical procedures in the abdominal cavity of the body require insufflating gases to raise the cavity wall away from vital organs, the procedure is usually initiated by use of a Verres needle through which a gas such as CO₂ is introduced into the body cavity, thereby creating a pneumoperitoneum. The gas provides a positive pressure which raises the inner body wall away from internal organs, thereby providing the surgeon with a region within which to operate and avoiding unnecessary contact with the organs by the instruments inserted through the cannula assembly. An obturator of the obturator assembly is inserted into the cannula assembly and used to puncture the abdominal wall. Following removal of the obturator assembly from the cannula assembly, laparoscopic or endoscopic surgical instruments may be inserted through the cannula assembly to perform surgery within the abdominal cavity.

Generally in the context of insufflatory surgical procedures, there are two sealing requirements for cannula assemblies. The first requirement is to provide a substantially fluid-tight seal when an instrument is not being introduced into or is not already present in the cannula. The second requirement is to provide a substantially fluid-tight seal when an instrument is being introduced into or is already present in the cannula.

In certain procedures, such as arthroscopic procedures, it is sometimes necessary to secure soft tissue to a selected bone surface either directly or indirectly via an implant typically called an anchor. These anchors are generally tied to sutures and the sutures are then tied to the soft tissue to hold it in place. Anchors may be used to attach soft tissue such as ligaments, tendons, muscles, etc., to a bone surface from which the soft tissue has become detached. Anchors may also be employed to secure soft tissue to supplementary attachment sites for reinforcement. During these kinds of procedures, surgeons may sometimes introduce multiple sutures through the cannula assembly of the trocar assembly. As a result, these sutures may tangle during the surgical procedure. In addition, identification of the suture may also be an issue.

In addition, in conjunction with an arthroscopic procedure, fluids such as saline, may be deposited into the surgical site to distend, e.g., the joint, and/or clean away debris thus presenting sealing concerns within the interior of the portal or cannula.

### SUMMARY

In accordance with the present disclosure, a seal assembly configured for use with a cannula assembly is provided. The seal assembly includes a housing, a suture retaining member, a slit seal member and an instrument seal member. The suture retaining member is disposed in mechanical cooperation with the housing and is configured to hold at least one suture to minimize the potential of suture entanglement. The suture retaining member may be disposed in mechanical cooperation with a proximal portion of the housing. The suture retaining member may have a plurality of suture parks disposed therethrough. Each suture park may be disposed adjacent to a slit defined through the suture retaining member.

The slit seal member is disposed in mechanical cooperation with the housing and is configured to create a substantially fluid-tight seal in the absence of a surgical instrument inserted therethrough. The slit seal member may be disposed in mechanical cooperation with the housing adjacent and distal to the suture retaining member. The seal assembly may also include two slit seal members wherein each slit seal member is disposed in mechanical cooperation with the housing.

The instrument seal member is disposed in mechanical cooperation with the housing and is configured to create a substantially fluid-tight seal around a surgical instrument inserted therethrough.

In accordance with the present disclosure, an access port is also provided, the access port including a housing and a seal assembly disposed within the housing. The seal assembly includes a first portion having a suture retaining member, a second portion having a slit seal member and a third portion having an instrument seal member. The housing may be configured to mechanically engage a cannula assembly.

In disclosed embodiments, the suture retaining member has a plurality of suture parks configured to hold sutures and is configured to help prevent the tangling of sutures. Each suture park may be disposed adjacent to a slit defined through the suture retaining member.

### BRIEF DESCRIPTION OF THE DRAWINGS

Various embodiments of the present disclosure are described herein with reference to the drawings wherein:

FIG. 1 is a perspective view of an access port including a suture retaining member, a slit seal member and an instrument seal member.

FIG. 2A is a top plan view of the suture retaining member of FIG. 1.

FIG. 2B is a top plan view of the slit seal member of FIG. 1.

FIG. 2C is a top plan view of the instrument seal member of FIG. 1.

FIG. 3 is a longitudinal cross-sectional view of the access port of FIGS. 1 and 2, attached to a cannula assembly and having an instrument disposed therethrough.

FIG. 4 is an exploded view of the suture retaining member, the slit seal member, and the instrument seal member of the access port of FIGS. 1-3.

### DETAILED DESCRIPTION

The access ports of the present disclosure, either alone or in combination with a cannula assembly, provide a substantially fluid-tight seal between a body cavity of a patient and the outside atmosphere. The access ports, or seal assemblies, of the present disclosure are configured to receive surgical instruments of varying diameter and are also configured to retain sutures therein and to help prevent the tangling of sutures. Various procedures contemplated include laparoscopic and arthroscopic procedures.

The access ports of the present disclosure contemplate the introduction of various types of instrumentation adapted for insertion through a trocar and/or cannula assembly while maintaining a substantially fluid-tight interface about the instrument to help preserve the atmospheric integrity of a surgical procedure from gas and/or fluid leakage. Examples of instrumentation include, but are not limited to, clip appliers, graspers, dissectors, retractors, staplers, laser probes, photographic devices, endoscopes and laparoscopes, tubes, and the like. Such instruments will collectively be referred to as “instruments” or “instrumentation.”

In addition to instrumentation, the access ports also allow the passage of one or more sutures therethrough. When several sutures are introduced into the subject's body through a typical access port, the sutures may tangle with each other. In addition, identification of the sutures also becomes an issue. To minimize the possibility of sutures tangling with one another and to assist the surgeon in identifying particular sutures and their respective functions, the access ports incorporate suture retaining members for holding the sutures in place.

In the following description, as is traditional, the term “proximal” refers to the portion of the device closer to the operator while the term “distal” refers to the portion of the device farther from the operator.

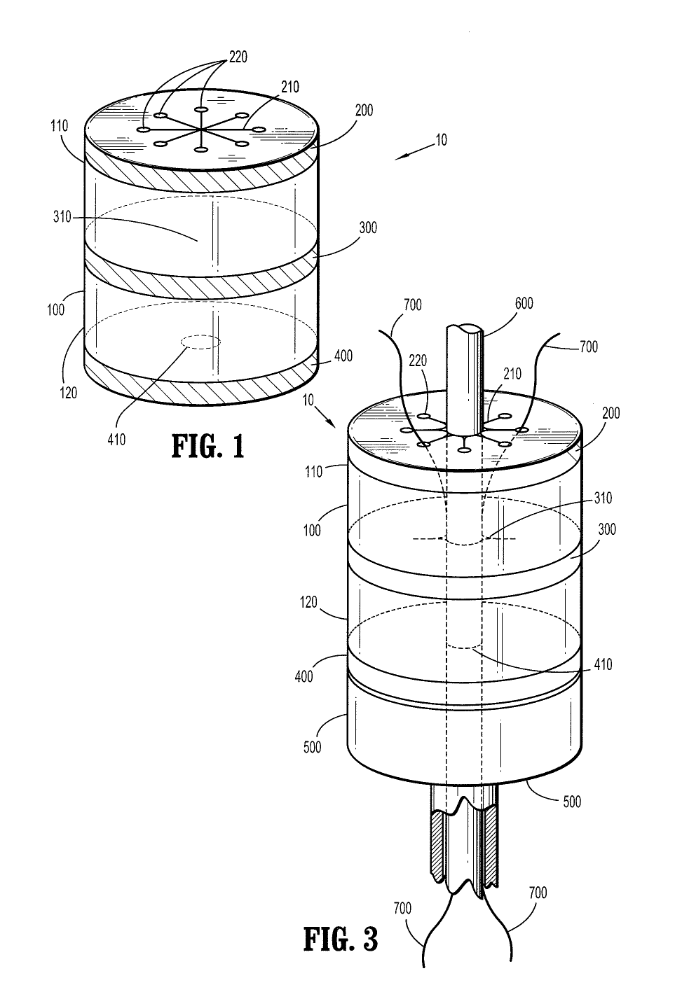

Referring now to the drawings, FIG. 1 illustrates a seal assembly 10 including a housing 100. Housing 100 of seal assembly 10 includes a suture retaining member 200 disposed in mechanical cooperation with housing 100, a slit seal member 300 disposed in mechanical cooperation with housing 100, and an instrument seal member 400 disposed in mechanical cooperation with housing 100. It is envisioned that at least one of suture retaining member 200, slit seal member 300 and instrument seal member 400 is fixedly attached to (e.g. monolithically formed with) housing 100.

In the embodiment shown in FIG. 1, suture retaining member 200 is disposed in mechanical cooperation with the proximal portion 110 of housing 100. The slit seal member 300 is disposed in mechanical cooperation with housing 100 adjacent and distal to the suture retaining member 200. The instrument seal member 400 is disposed in mechanical cooperation with the distal portion 120 of housing 100 adjacent and distal to slit seal member 300.

It is envisioned that a second slit seal member (not shown) may also be included within housing 100 such that the slit seal members are adjacent each other. The second slit seal member would function in substantially the same manner as the first slit seal member 300 and would not significantly alter the operation of the seal assembly 10 as described below.

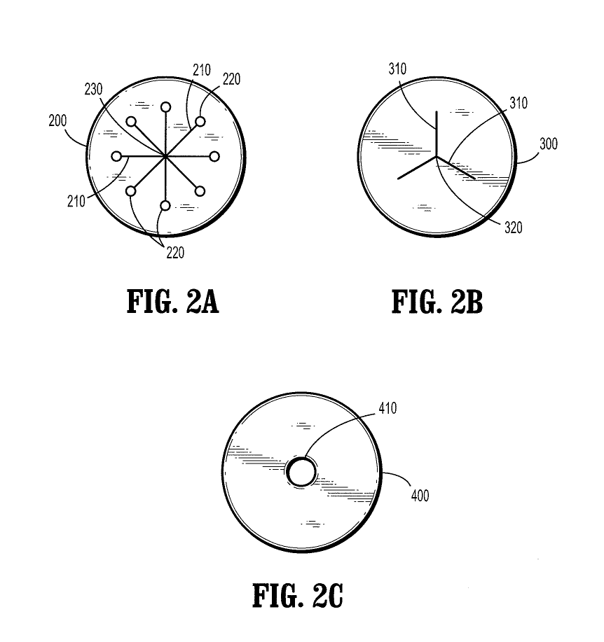

FIGS. 2A-C are top plan views of the suture retaining member 200, the slit seal member 300 and the instrument seal member 400, respectively. Suture retaining member 200 includes a plurality of suture parks 220 configured to hold sutures 700 (see FIG. 3) and configured to help prevent the tangling of sutures 700. The suture parks 220 are disposed adjacent to slits 210 defined in suture retaining member 200. Slits 210 allow for the passage of sutures and/or surgical instrumentation therethrough. Slits 210 are configured such that at rest, slits 210 are in a closed position. Slits 210 may intersect or meet at intersection point “K” which may or may not be coincident with the longitudinal axis. Slits 210 communicate which may facilitate mounting and/or positioning of sutures relative to suture retainer member 210. As shown in FIG. 2A, the slits 210 of suture retaining member 200 extend radially from a center 230 of suture retaining member 200. Each slit 210 extends into a suture park 220. The suture parks 220 are configured such that a suture disposed therethrough will remain in that respective suture park 220. The operation of suture retaining member 200 will be discussed in more detail below.

The embodiment of FIG. 2A shows eight slits 210 and eight corresponding suture parks 220. However, it is envisioned and within the scope of the present disclosure that more or fewer slits 210 and/or suture parks 220 may be provided.

FIG. 2B is a top plan view of slit seal member 300. Slit seal member 300 includes a plurality of slits 310 extending from a center 320 of slit seal member 300. While three slits 310 are shown, it is envisioned and within the scope of the present disclosure that more or fewer slits 310 may be provided. Slits 310 allow for the passage of sutures and/or surgical instrumentation therethrough. In the absence of a surgical instrument disposed through seal assembly 10, slit seal member 300 including slits 310 is in an at-rest or closed position. Slit seal member 300 is configured to create a substantially fluid-tight seal in the absence of an instrument passing therethrough. The operation of slit seal member 300 will be discussed in more detail below.

FIG. 2C is a top plan view of instrument seal member 400. In the illustrated embodiments, instrument seal member 400 includes an aperture 410 configured to create a fluid-tight seal around an instrument inserted therethrough. The operation of instrument seal member 400 will be discussed in more detail below

Referring now to FIG. 3, seal assembly 10 is shown in mechanical cooperation with cannula assembly 500. A distal end 120 of housing 100 is shown monolithically formed with a cannula assembly 500. Seal assembly 10 may also be configured to mechanically engage cannula assembly 500 in a variety of ways including, but not limited to, through a bayonet lock or threaded connection. Cannula assembly 500 may include a second seal (not shown) which provides a substantially fluid-tight seal in the absence of a surgical instrument passing therethrough.

FIG. 3 illustrates instrument 600 passing through seal assembly 10 and into cannula assembly 500. Individual sutures 700 are disposed through seal assembly 10 and through suture parks 220. Instrument 600 passes through slits 210 of suture retaining member 200, slits 310 of slit seal member 300, and aperture 410 of instrument seal member 400. Instrument 600 extends into cannula assembly 500.

The operation of seal assembly 10 will now be described in detail, with reference to FIGS. 1-3. Referring to FIG. 1, seal assembly 10 is provided. Seal assembly 10 may be mounted onto cannula assembly 500 (FIG. 3). Sutures 700 may be disposed through slits 210 of suture retaining member 200, through slits 310 of slit seal member 300, through aperture 410 of instrument seal member 400 and into cannula assembly 500.

Once disposed as described above, a suture 700 may be pulled or dragged until disposed through a suture park 220. Sutures 700 disposed through suture parks 220 are retained in suture parks 220, thereby minimizing tangling of multiple sutures 700 disposed within housing 100 of seal assembly 10. In addition, placement of sutures 700 within suture parks 220 will assist in identifying the respective sutures and their functioning. Positioning of sutures 700, as described above, may be repeated until each suture 700 is disposed in a suture park 220. Suture parks 220 are spaced apart and configured such that the likelihood of entanglement between a suture 700 disposed through a suture park 220 and another suture 700 disposed through another suture park 220 is reduced. Suture retaining member 200 may be fabricated from an elastomeric or resilient material.

An instrument 600 may be introduced into seal assembly 10 as follows. The instrument 600 is first inserted through slits 210 of suture retaining member 200. Slits 210 expand from their at-rest position to accommodate instrument 600 therein. Slits 210 allow instrument 600 to pass therethrough, without displacing sutures 700 from suture parks 220. As instrument 600 is farther advanced into access port 10, instrument 600 passes through slits 310 of slit seal member 300. Slits 310 expand to accept instrument 600. As instrument 600 is advanced even farther, instrument 600 passes through aperture 410 of instrument seal member 400. Aperture 410 creates a substantially fluid-tight seal around instrument 600 when disposed therethrough.

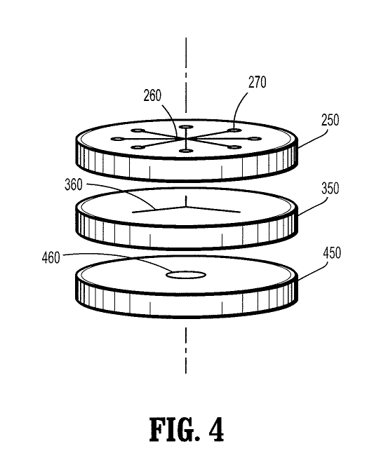

In the embodiment shown in FIG. 4, an access port 20 of the present disclosure includes first portion 250, second portion 350 and third portion 450. Access port 20 is different from seal assembly 10 in that the three portions 250, 350 and 450 of access port 20 are integrally molded to form a seal assembly which is disposed within a housing. Seal assembly 10 includes a housing within which a suture retaining member, a slit seal member, and an instrument seal member are disposed, and not integrally formed therewith.

First portion 250 of access port 20 is shown positioned proximal to and adjacent second portion 350. Second portion 350 is shown positioned proximal to and adjacent third portion 450. First portion 250 includes a suture retaining member including slits 260 and suture parks 270. Slits 260 and suture parks 270 of first portion 250 operate in substantially the same manner as slits 210 and suture parks 220 of suture retaining member 200, as described above.

Second portion 350 includes a slit seal member including slits 360. Slits 360 of second portion 350 operate in substantially the same manner as slits 310 of slit seal member 300, as described above.

Third portion 450 includes an instrument seal member including an aperture 460. Aperture 460 operates in substantially the same manner as instrument seal 410 of instrument seal member 400, as described above.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A seal assembly configured for use with a cannula assembly, the seal assembly comprising:
a housing;
a suture retaining member disposed in mechanical cooperation with the housing, the suture retaining member configured to hold at least one suture and configured to help prevent the tangling of sutures;
a slit seal member disposed in mechanical cooperation with the housing, the slit seal member configured to create a substantially fluid-tight seal in the absence of a surgical instrument inserted therethrough; and
an instrument seal member disposed in mechanical cooperation with the housing, the instrument seal member configured to create a substantially fluid-tight seal around a surgical instrument inserted therethrough.

2. The seal assembly of claim 1, wherein the seal assembly comprises two slit seal members and wherein each slit seal member is disposed in mechanical cooperation with the housing.

3. The seal assembly of claim 1, wherein the suture retaining member is disposed in mechanical cooperation with a proximal portion of the housing.

4. The seal assembly of claim 1, wherein the slit seal member is disposed in mechanical cooperation with the housing adjacent and distal to the suture retaining member.

5. The seal assembly of claim 1, wherein the suture retaining member has a plurality of suture parks disposed therethrough.

6. The seal assembly of claim 5, wherein each suture park is disposed adjacent to a slit defined through the suture retaining member.

7. The seal assembly of claim 1, wherein a distal portion of the housing is configured to mechanically engage a cannula assembly.

8. The seal assembly of claim 1, wherein at least one of the suture retaining member, the slit seal member and the instrument seal member is fixedly attached to the housing.

9. The seal assembly of claim 1, wherein each of the suture retaining member, the slit seal member and the instrument seal member is fixedly attached to the housing.

10. An access port comprising:
a housing; and
a seal assembly disposed within the housing, the seal assembly including:
a first portion including a suture retaining member;
a second portion including a slit seal member; and
a third portion including an instrument seal member.

11. The access port of claim 10, wherein the housing is configured to mechanically engage a cannula assembly.

12. The access port of claim 10, wherein the suture retaining member has a plurality of suture parks configured to hold sutures and is configured to help prevent the tangling of sutures.

13. The access port of claim 12, wherein each suture park is disposed adjacent to a slit defined through the suture retaining member.
